(19) United States
(12) Patent Application Publication
(10) Pub. No.: US 2004/0053308 A1
(43) Pub. Date: Mar. 18, 2004

(54) Probe immobilized substrate and method for manufacturing the same, and analytical method

(75) Inventor: Kumi Nakamura, Kanagawa (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, TOKYO (JP)

(21) Appl. No.: 10/463,549

(22) Filed: Jun. 18, 2003

(30) Foreign Application Priority Data
Jun. 28, 2002 (JP) 190009/2002
Jun. 28, 2002 (JP) 189836/2002

(51) Int. Cl. B05D 3/00; C12Q 1/68; C12M 1/34

(52) U.S. Cl. 435/6; 435/287.2; 427/2.11

(57) ABSTRACT

The present invention provides a probe immobilized substrate and a method for manufacturing the same, wherein the probe is able to be analyzed without any problems using various surface analysis methods, sampling inspection loss becomes minimum, and target substances are able to be promptly and cheaply detected and quantified.

### BACKGROUND OF THE INVENTION

1. Field of the Invention

The present invention relates to a probe immobilized substrate and a method for manufacturing the same, and an analytical method.

2. Description of the Related Art

So-called bio-tips such as DNA chips and protein chips have been utilized for genome analysis and gene expression analysis, and the results of the analysis are expected to provide crucial guidelines for diagnosis of cancers, hereditary diseases, life style diseases and infectious diseases, forecast of prognosis and determination of therapeutic strategies.

Research and development related to detection and quantification of target substances using solid phase probe arrays have became active in resent years. For example, U.S. Pat. No. 5,445,936 has disclosed a solid phase oligonucleotide array prepared using a photolithographic method. On the other hand, U.S. Pat. No. 5,688,642 has disclosed a method for preparing a solid phase DNA probe using an ink-jet method.

While the bio-chips are usually prepared by any one of these methods, it is important to determine the quantity of the probe, or the quantity of biological substances in this case, present in each matrix in order to guarantee quantitative accuracy and reproducibility when these bio-chips are used for the purposes as described above. It is also important to know in what configurations (shape, size and state) of the matrix the probes are actually present on the chip. However, since the probe is formed as a monolayer of molecules in principle, a quite highly sensitive technique is required for such surface analysis.

It is considered to be crucial for preventing medical malpractice to inspect defects, if any, of the probe immobilized substrate that deals with gene information during the manufacturing process as well as to inspect before shipment, as is considered in other medical products.

It is also important to know which prove of a plurality of different probes constituting the array has reacted with the target substance, when the target substrate is detected and quantified using the probe immobilized substrate.

Fluorescence methods are usually used for these inspection methods. In these methods, a probe array immobilized on the substrate and target substances containing fluorescent substances are coupled to detect fluorescence. Examples of the fluorescent substance include Cy3 and Cy5, and a confocal laser apparatus is commercially available for the scanner for sensing fluorescence.

When the fluorescence method is used for inspections in the manufacturing process or before shipping, a part of the probe immobilized substrates manufactured are sampled, a fluorescent substance is coupled to a standard target substance that is previously known to specifically bind to a probe array disposed on the substrate by allowing the standard substance to contact the probe array, excess target substances are removed after an appropriate reaction time, and fluorescence is detected with the scanner.

When the inspection method using the fluorescence method is applied to the solid phase probe manufactured by the ink-jet method, it may be sometimes difficult to locate the position of each probe relative to a specific position on the substrate due to the difference of the manufacturing apparatus as compared with the lithographic method. Accordingly, Japanese Patent Laid-Open No. 2000-270896 discloses a probe array and a method for manufacturing the same, wherein a pigment or a fluorescent pigment as a marker is disposed around the probe, and the probe is located from the position of the marker.

Luminous positions when the standard target substance is bound to the probe can be discriminated by using this probe array by detecting the pigment (the fluorescent pigment) around the probe.

The inventors of the invention have found the following problems through further studies on the technique for detecting and quantifying the target substance using the probe array manufactured by an ink-jet method.

The marker substances such as the pigment and fluorescent pigment described in Japanese Patent Laid-Open No. 2000-270896 cannot be sometimes discriminated when analytical methods except the fluorescence method and optical microscopy are used.

It is also important to confirm for inspections in the manufacturing process and before shipping whether the probe arrays prepared by the ink-jet method are formed at prescribed positions or not, or whether the probe arrays are correctly formed or not. However, although the position of the probe can be confirmed by detecting droplets immediately after dripping the droplets of the probe on the substrate, the relative positions of the probes cannot be confirmed by using a conventional optical microscope and fluorescence microscope after the solution containing the probe has been washed away after completing the coupling reaction of the probe with the substrate in the manufacturing process. It is also impossible to confirm whether the probes have been disposed on the substrate with a desired shape and quantity using the optical microscope. In other words, the probe formed on the substrate cannot be observed with the optical microscope or fluorescence microscope since the solution containing the probe is inherently colorless and transparent, and the probe left behind on the substrate after the solution has been washed away is quite minute.

Japanese Patent Laid-Open No. 2000-270896 discloses a method for observing the positions and shapes of the probes by permitting individual probe array to emit a light, wherein the probe array is manufactured by disposing the fluorescent pigment or pigment around the probe, a part of the probe arrays are sampled, and a fluorescent substance is bound to a target substance (to which a fluorescent substance is bound) that is previously known to specifically bind to each probe array. However, since the standard target substance is bound to the probe for detecting by the fluorescence method, the probe immobilized substrate after inspection cannot be delivered as a product. Since the object of the probe immobilized substrate is to detect many kinds of the target substances using one sheet of the probe immobilized substrate, a number of probes are present on the probe immobilized substrate. Accordingly, large decrease of the number of the products that is ready for shipping by sampling by inspection of every kinds of the probes gives a great economical loss to the manufacturers.

For the reasons above, different inspection methods have been desired in the manufacturing process. The inventors of the invention have found, through intensive efforts and studies, that surface analysis methods including scanning electron microscopy, photoelectron spectroscopy, atomic force microscopy and time-of-flight secondary ion mass spectrometry (abbreviated as TOF-SIMS hereinafter) are effective for inspecting the probe.

However, it was a problem that the position of the probe cannot be located on the substrate during analysis, since signals emitted from the probe array as well as from markers comprising the pigment or fluorescent pigment are weak when the probe immobilized substrate is observed by irradiating with a radiation flux such as an electron beam, X-ray or an ion beam in this analytical method.

While the substrate for the probe immobilized substrate is not particularly restricted so long as the substances that constitutes the probe are able to be immobilized, and detection of the target substance is not inhibited, an example is a glass substrate. While a silicon substrate, metal substrate and resin substrate, or respective substrates after an appropriate surface treatment, are also available, the glass substrate is frequently used considering advantages and convenience that various methods known in the art can be used for cleaning and surface treatment while the substrate itself is readily available.

However, it was found to be a problem that the analysis is inhibited by electrification as a result of irradiation of the radiation flux such as the electron beam, X-ray and ion beam when the analytical method as described above is employed for the glass substrate.

### SUMMARY OF THE INVENTION

The object of the invention for solving the problems above is to provide a probe immobilized substrate and a method for manufacturing the same, whereby the probes can be promptly located by using a surface analysis method such as scanning electron microscopy, photoelectron spectroscopy, atomic force microscopy and a TOF-SIMS method.

Another object of the invention for solving the technical problems above provides a probe immobilized substrate and a method for manufacturing the same, wherein the probe can be analyzed without any problems using a surface analysis method such as scanning electron microscopy, X-ray photoelectron spectroscopy and a TOF-SIMS method for analyzing the prove immobilized substrate prepared on an insulating substrate, or on a substrate having an insulating layer on a conductive substrate.

A different object of the present invention is to provide a prove immobilized substrate and a method for manufacturing the same, wherein the probe can be shipped with a minimum loss by sampling inspection.

A further different object of the invention is to provide a prove immobilized substrate and a method for manufacturing the same, wherein target substances can be promptly and cheaply detected and quantified.

A further different object of the invention is to provide a method for promptly and cheaply quantifying target substances in a test sample.

A further different object of the invention is to provide a method for promptly inspecting the probe array with a minimum loss in the manufacturing process and inspection process after manufacturing the probe array that is able to detect and quantify the target substance.

Accordingly, the invention provides a probe immobilized substrate on which a probe capable of specifically binding to a target substance is immobilized to a first position on the surface of the substrate. A metal or a metal compound is disposed at a second position capable of locating the first position with a special relation to the first position.

The invention for attaining the objects above provides a method for manufacturing a probe immobilized substrate comprising the steps of: supplying a liquid containing a probe capable of specifically binding to a target substance on a first position of the surface of a substrate; and disposing a metal or a metal compound at a second position capable of locating the first position with a special relation to the first position. The probe is supplied after disposing the metal or the metal compound.

The invention also provides a method for manufacturing a probe immobilized substrate comprising the steps of: supplying a liquid containing a probe capable of specifically binding to a target substance on a first position of the surface of a substrate; and disposing a metal or a metal compound at a second position capable of locating the first position with a special relation to the first position. The metal or the metal compound is supplied to the surface of the probe immobilized substrate at the same time as supplying the liquid containing the probe onto the surface of the substrate.

The invention also provides a method for analyzing a probe array having probes capable of specifically binding to target substances that may be potentially contained in a test sample as a plurality of spots being independent with each other. The method comprises the steps of: disposing a marker comprising the metal or metal compound so as to be able to locate each spot on the surface of the substrate; and locating each spot based on the position of the marker.

The invention also provides a method for detecting a probe and a target material, if any, in any one of the spots by allowing each spot of the probe array having the probes that specifically bind to a target substance that may be potentially contained in a test sample as a plurality of mutually independent spots to contact the test sample. The method comprises the step of locating the spot containing a reaction product based on a metal or a metal compound when the reaction product is detected to exist.

The invention is further featured as follows.

The invention provides a probe immobilized substrate comprising a probe capable of specifically binding to a target substance immobilized on the surface of a substrate having at least an insulating surface. A conductive layer is flush with at least the surface of the probe.

The invention for attaining the objects above provides a method for manufacturing the probe immobilized substrate comprising the steps of supplying a liquid having probes capable of specifically binding to target substances, and disposing a conductive layer on the surface of the substrate, wherein the probe is supplied after disposing the conductive layer.

In a different aspect, the invention provides a method for manufacturing the probe immobilized substrate comprising the steps of supplying a liquid having probes capable of specifically binding to target substances, and disposing a conductive layer on the surface of the substrate, wherein the conductive layer is disposed at the same time as supplying the liquid containing the probes onto the surface of the substrate.

In a further different aspect, the invention provides a method for analyzing a probe array comprising probes capable of specifically binding to a target substance that may be potentially contained in a test sample as a plurality of spots being independent with each other, wherein the probe is analyzed after disposing the conductive layer on the surface of the substrate followed by electrically connecting the conductive layer to the ground.

Further objects, features and advantages of the present invention will become apparent from the following description of the preferred embodiments (with reference to the attached drawings).

### BRIEF DESCRIPTION OF THE DRAWINGS

FIG. 1 shows an embodiment of the invention.

FIG. 2 shows another embodiment of the invention.

FIG. 3 shows a different embodiment of the invention.

FIG. 4 shows an example of the invention

FIG. 5 shows Example 3 of the invention.

FIG. 6 shows an embodiment of the invention.

FIG. 7 shows Example 6 of the invention.

FIG. 8 shows Example 9 of the invention.

FIG. 9 shows Example 8 of the invention.

FIG. 10 shows an embodiment of the invention

FIG. 11 shows a different embodiment of the invention.

FIG. 12 shows Example 10 of the invention.

### DESCRIPTION OF THE PREFERRED EMBODIMENTS

The invention will be described in detail hereinafter with reference to the examples.

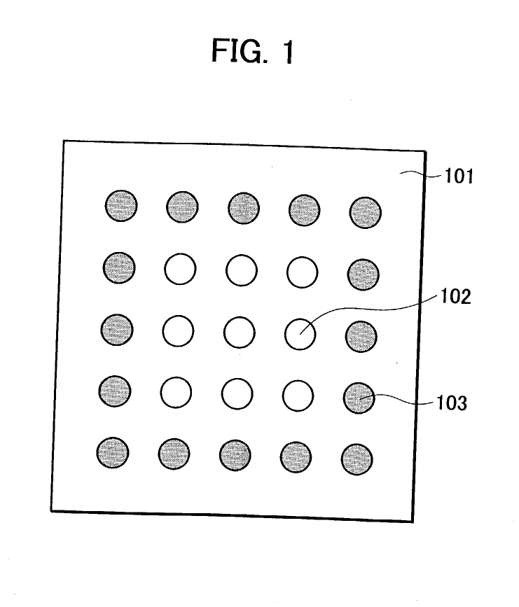

FIG. 1 shows a plane view of a probe array in which spots of a plurality of probes having different base sequences with each other are immobilized on a substrate 101. The reference numeral 102 denotes the spot of the probe, and the reference numeral 103 denotes a spot of a metal or metal compound, which are disposed at a position capable of locating each spot 102 of the probe. Actually, the spots 103 are arranged so as to correspond to each column and row of the spots 102 of the probe, and each spot 102 can be located by this arrangement of the spots 103.

While the substrate 101 is not particularly restricted so long as the material constituting the probe is able to be immobilized on the substrate and does not inhibit detection of target substances, an example of the substrate is a glass substrate. However, a silicon substrate, metal substrate and resin substrate, or those after an appropriate surface treatment, are also available. Use of the glass substrate is advantageous and favorable, since various methods known in the art can be used for cleaning and surface treatment while the substrate itself is readily available.

The probe 102 used in the invention is able to specifically bind to the target substance, and a target for detecting that the probe is bound to the target substance may be immobilized together. A representative example of the probe is a single strand nucleic acid, which may a single strand DNA, single strand RNA or single strand PNA (peptide nucleic acid). These probes may be appropriately selected from those known in the art depending on the kinds of the label substances.

The metal or metal compound to be used for the spot 103 of the metal or metal compound is not particularly restricted, and those used in conventional semiconductor processes may be used.

While examples of the metal or metal compound to be used for the invention include metals such as Au, Ag, Cu, Ni, Co, Cr, Al, Ta, Pt, Pd, Zn and Sn, and compounds thereof, they are not particularly restricted.

The metals and metal compounds are suitable as markers for indicating the position of the probe array spot 102, since spots comprising these metals and metal compounds are detectable with an optical microscope or a stereoscopic microscope when the spots are colored by irradiating with, for example, a visible light.

The layer of the metal or metal compound, for example a fine particle layer, comprises particles having a sufficiently small diameter of 1 μm or less, for example a mean diameter of 100 nm or less. Accordingly, the metal or metal compound is able to be uniformly deposited as a thin layer, and is favorable for forming an arbitrary configuration of the metal layer for assessing the probe array.

The spots 102 of the probe, and the spots 103 of the metal or metal compound can be formed by applying a liquid containing the probe, and a liquid containing the metal or metal compound, on the substrate using, for example, an ink-jet method.

The ink-jet method is suitable since fine probes can be accurately discharged as fine droplets (several pico-liter to several tens nano-liter). While practically used ink-jet methods today include an ink-jet method using a piezoelectric element and a thermal jet method using a thermal element, either method may be used in the invention.

It is preferable to adjust the composition of the liquid for applying the spots 103 of the metal or metal compound on the substrate by the ink-jet method, in order to allow the droplets to stay at prescribed positions without unnecessarily spreading on the substrate. Since the droplets supplied on the surface of the substrate are fine when the ink-jet method is used, it is effective to allow a moisture retaining agent to be contained in the discharged liquid for preventing the droplets from being evaporated after discharge. Such moisture retaining components and surface tension adjusting components are important particularly in the thermal jet method, since the temperature increases during discharge. A favorably used solvent for supplying the probes on the surface of the substrate contains 5 to 10 wt % of urea, 5 to 10 wt % of glycerin, 5 to 10 wt % of thiodiglycol and 1 wt % of acetylene alcohol.

While any methods may be used for preparing the solution containing the metal or metal compound for use in applying the spots 103 of the metal or metal compound by the ink-jet method so long as the method is able to stably and continuously discharge the fine droplets, the solution is favorably prepared by dissolving an organometallic complex or metallic colloid in an appropriate solvent, since a fine article layer is preferably formed after deposition.

Examples of center metals for use in the organometallic complex include Pt, Pd, Ru, Au, Ag, Cu, Cr, Ta, Fe, W, Zn and Sn. Water or an organic solvent is used for the solvent depending on the solubility of the organometallic complex. However, water is preferable considering to reduce the cost and environmental protection, and the solvent is favorably prepared by dissolving the moisture retaining component and surface tension adjusting component together with a metal in water.

For example, metals such as Pd, Au, Ag and Pt, and SnO₂ are used for the metallic colloid. Water or an organic solvent is used as the solvent depending on the method for preparing the colloid. Examples of the organic solvent include hexane and pentaethyleneglycol decylether.

The spots 103 of the metal or metal compound may be either formed in the same step as forming the spots 102 of the probe, or formed in advance to forming the probe.

When the spots 103 of the metal or metal compound are formed by the ink-jet method in advance to forming the probe, water may be used as a principal solvent for the solution containing the metal or metal compound even when the substrate is subjected to a step for washing with water after a coupling reaction between the probe and substrate, since the spots 103 of the metal or metal compound have been already formed.

Practically, an aqueous solution of a selected organometallic complex or metallic colloid, or a solution containing an appropriate moisture retaining agent and surface tension retaining agent in this aqueous solution may be used.

The spots 103 are formed by spontaneous drying, or by heating the substrate for evaporating the solvent, after discharging the spots 103 of the metal or metal compound. Organic components in the complex are removed by drying the substrate on which the spots 103 have been formed at a high temperature of 200 to 400° C., thereby forming the spots 103 comprising the fine particles of the metal or metal compound.

The spots 102 of the probe are formed thereafter by the same method as simultaneously forming the spots as described above by discharging the solution containing the probe to allow it to react with the substrate.

Various kinds of the metal or metal complex can be used for the marker spots 103 by using the method for forming the spots of the probe.

The solution containing the metal or metal compound should be devised so that the spots 103 of the metal or metal complex are not removed in the cleaning step when the spots 103 of the solution containing the metal or metal compound are formed in the same step as forming the spots 102 of the probe, considering the cleaning step applied after a reaction of the probe array and target substance after forming both kinds of spots.

In an effective method, the proportion of the moisture retaining agent is reduced to be smaller than the proportion of the solution containing the probe in the solution containing the metal or metal compound. Only the solvent of the solution containing the metal or metal compound is dried during the coupling reaction time between the solution containing the probe and the substrate in order to prevent the spots 103 from being removed in the cleaning step. The proportion of the moisture retaining agent is appropriately selected depending on the principal solvent of the ingredients except the moisture retaining agent.

When the spots 103 of the metal or metal compound are formed in the same step as forming the spots 102 of the prove, the probe immobilized substrate can be more promptly manufactured by omitting positioning steps between the rows or columns of the probe and the spots 103 of the metal or metal compound.

Since the spots of the metal or metal compound can be readily formed as metal layers or metal compound layers at arbitrary positions using the ink-jet method as described above, the spots are useful for in-line analysis in the manufacturing spots and for analysis for inspection before shipping.

These spots may be used as markers by providing a circular single spot or a plurality of circular spots, or may be formed into a different shape by combining a plurality of spots. For example, the spots may be provided for addressing columns and rows.

The method for analyzing the probe immobilized substrate of the invention will be described hereinafter.

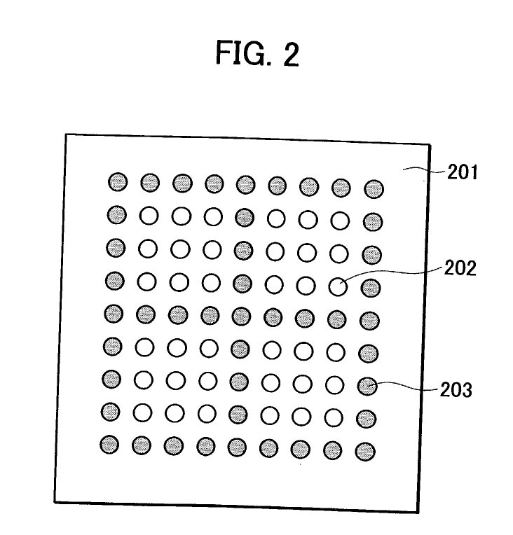

FIG. 2 shows another embodiment of the probe immobilized substrate of the invention. The reference numeral 201 denotes a substrate, the reference numeral 202 denotes a probe array, and the reference numeral 203 denotes a metal or metal compound.

As an example of the analytical method of the invention, the probe immobilized substrate is analyzed with an X-ray photoelectron spectrometer. At first, the probe immobilized substrate of the invention is introduced into a chamber.

While the substrate is observed with an attached CCD camera for determining measuring positions on the substrate introduced into the chamber, it was quite difficult to locate the probe array in the conventional probe immobilized substrate comprising only the probe array disposed thereon, because the amount of the probe array 202 was minute while being transparent and colorless. However, since the spots 203 of the metal or metal compound are aligned on the same lines as the X and Y axes, the measuring points can be located by tracing the positions of the metal or metal compound.

For example, while a metal cover for preventing electrification should be usually provided on the substrate for using a monochromatic X-ray source as the X-ray source when the measuring area is as small as 1 mm or less on a insulator such as the glass substrate, the metal cover can be readily coated on the desired probe positions by positioning the probe array.

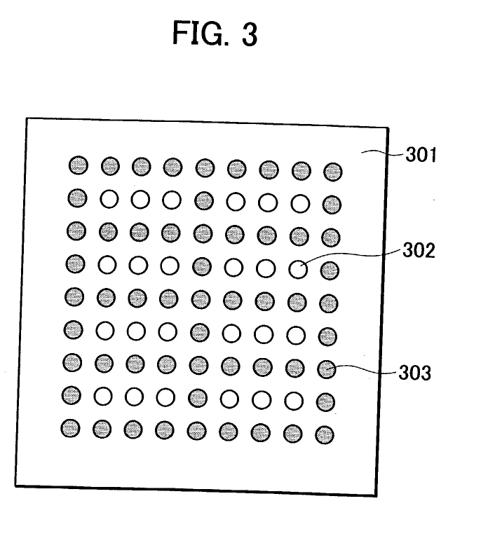

FIG. 3 shows a different embodiment of the probe immobilized substrate of the invention. The reference numeral 301 denotes a Si substrate, the reference numeral 302 denotes a probe array, and the reference 303 numeral denote a metal or metal compound.

In an example of the analytical method of the invention, the probe immobilized substrate of the invention is analyzed with a scanning electron microscope. At first, the probe immobilized substrate of the invention is introduced into a chamber.

While the overall configuration of the substrate is confirmed by a low magnification mode for locating the measuring point on the substrate introduced into the chamber, it is quite difficult and takes a long period of time to locate each probe array by the low magnification mode in the conventional probe immobilized substrate on which only the probe array is disposed, since the probe array is very thin and is made of an organic compound having a low secondary electron emission efficiency. However, the measuring points can be promptly and easily located by tracing the positions of the metal or metal compound spots, since the spots 303 of the metal or metal compound are aligned on the same lines as the X and Y axes of the probe array in the probe immobilized substrate of the invention. Further fine structures of the array can be also observed by conventional optical electron microscopy or by the fluorescence method.

While X-ray photoelectron spectroscopy and scanning electron microscopy have been exemplified for the analytical method using the probe immobilized substrate of the invention, the methods are not restricted thereto. Various surface analysis methods such as an electron microanalyzer, Auger electron spectroscopy, SIMS and electron microscopy are also useful as the analytical methods of the probe immobilized substrate.

While analysis of the probe array has been described in the embodiment above, the method is not restricted to inspection in the manufacturing process of the probe array. The method is also effective for detecting the target substance by analyzing after a coupling reaction between the array and test samples.

The second embodiment of the invention will be described in detail hereinafter with reference to the drawings.

FIG. 9 is a plane view of a probe array on which a plurality of probes having different base sequences with each other are immobilized on an insulating substrate 401 as spots. The reference numeral 402 denotes probe array spots, and the reference numeral 403 denotes a conductive layer, which is electrically connected to a ground potential during the analysis as shown in the drawing.

While the substrate 401 is not particularly restricted so long as it is able to immobilize the substance constituting the probe and does not interfere with detection of the target substance, an example thereof is a glass substrate. Other substrates available include those subjected to appropriate surface treatments. The method of the invention is also applicable to the substrates prepared by depositing an insulation layer such a SiO₂ layer by sputtering, printing or heat oxidation on a conductive substrate such as a silicon substrate or metal substrate

Use of the glass substrate as the substrate is advantageous and favorable, since the substrate can be cleaned and surface-treated by various methods known in the art while the substrate itself is readily available.

The probe array spots 402 to be used in the invention can be coupled with the target substance, and a label for detecting coupling of the target substance may be immobilized to each spot, if necessary. Representative examples of the substance to be used as the prove include a single strand nucleic acid and protein antibody, and examples of the single strand nucleic acid include a single strand DNA, single strand RNA and single strand PNA (peptide nucleic acid. The probe may be appropriately selected from those known in the art, and may be used depending on the kinds of the label substances.

The materials to be used for the conductive layer 403 include a metal, metal compound and carbon. Actually, the metal or metal compound to be used in the invention may be a material that renders the volume resistivity of the conductive layer 403 to be 1×10⁷ Ω·cm, more preferably 1×10⁵ Ω·cm. While examples of the metal include Au, Ag, Cu, Ni, Co, Cr, Al, Ta, Pt, Pd, Zn and Sn, and compounds thereof, they are not restricted thereto, and materials used in the conventional semiconductor process may be used so long as the materials satisfy the volume resistivity described above.

Using the conductive layer 403 satisfying the conditions above permits electrification of the substrate to be prevented as will be described below when the probe immobilized substrate on the insulator substrate is analyzed.

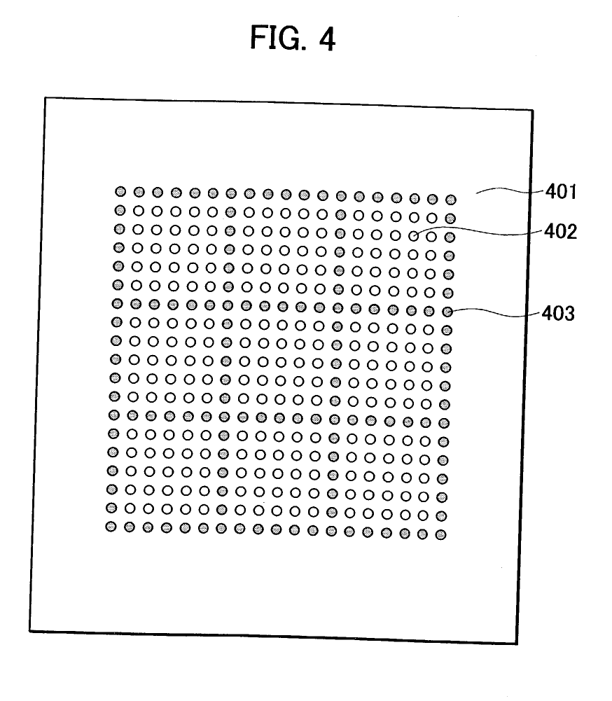

While the conductive layer 403 may be formed at least to be flush with the probe, it is preferably formed at a different level from the prove, since wettability of the conductive layer is changed when it is deposited at the position in contact with the prove to make the probe preparation conditions to be changed for every conductive layers. For example, it is preferable to deposit the conductive layer around the probe as shown in FIG. 4.

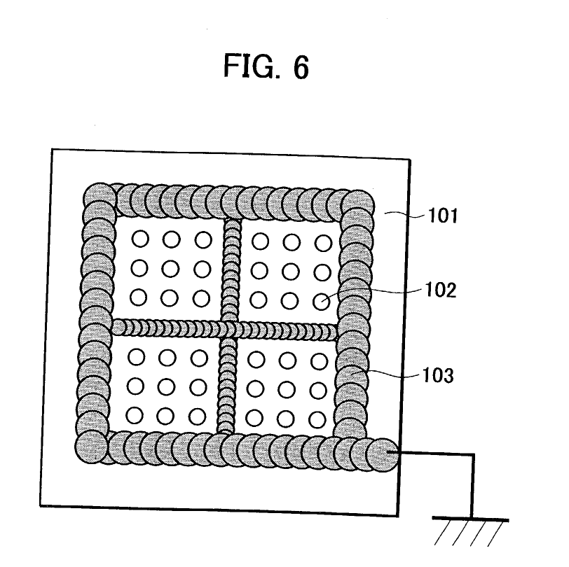

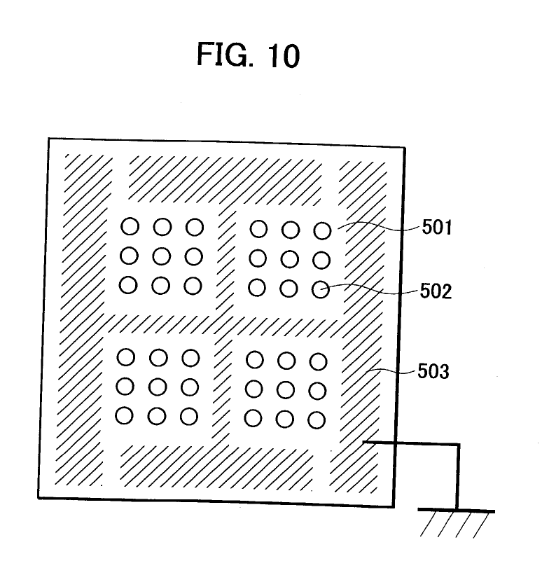

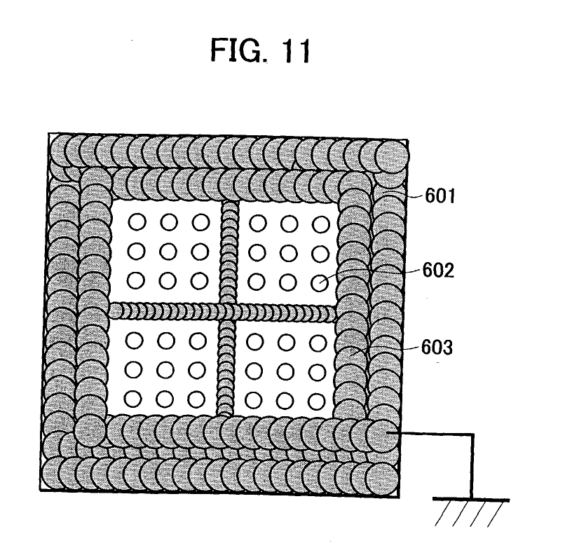

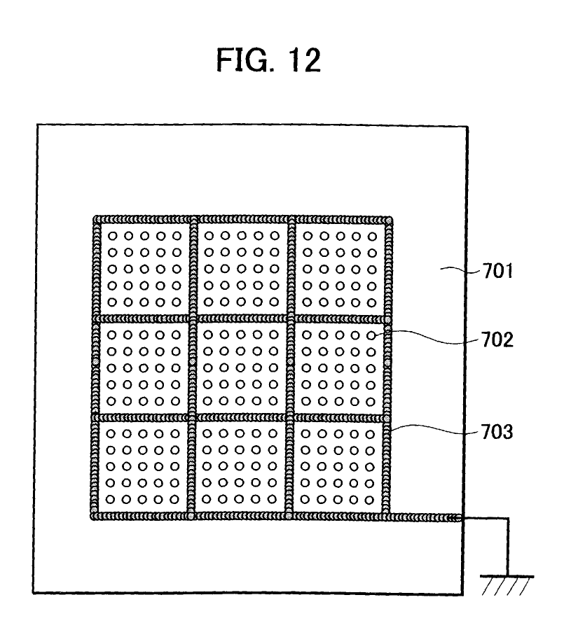

Since the insulation surface around the probe is preferably covered with the conductive layer to be as close as possible, the surface on which the prove is not immobilized is preferably covered with the conductive layer as shown in FIGS. 9 and 11. However, the configuration of the conductive layer is not restricted thereto, and the insulation surface may be exposed as shown in FIGS. 6 and 10 so long as the conductive layer connected to the ground is formed around the probe.

The configuration of the conductive layer 403 may be a continuous layer as shown in FIGS. 9 and 11, or the continuous layer may be formed by overlapping fine spots as shown in FIGS. 6 and 11.

While the method for depositing the conductive layer 403 to be used in the invention is not particularly restricted so long as the conductive layer is provided at desired positions, examples of the method include a vacuum deposition method, sputtering method, printing method and ink-jet method.

The conductive layer may be patterned into a desired shape by overlaying a metal mask when the conductive layer is otherwise formed on the entire surface of the substrate by the vacuum deposition method and sputtering method.

The liquid containing the metal or metal compound may be patterned on the substrate by the ink-jet method.

The probe array spots 402 to be used in the invention may be also patterned by the applying the liquid containing the probe on the substrate using the ink-jet method.

The ink-jet method is suitable for the object of the invention, since the method is able to discharge minute droplets (several picoliter to several tens nanometer) to enable minute probes to be formed. While the currently available ink-jet method includes a method using a piezoelectric element and a method using a thermal element, either method may be used in the invention.

The metal or metal compound as a material for forming the conductive layer by the ink-jet method has a particle diameter of sufficiently smaller than 1 μm, practically has a mean particle diameter of 100 nm or less. Accordingly, the metal or metal compound can be uniformly deposited as a thin layer, which is suitable for evaluation of the probe array since an arbitrary configuration of the metal layer is formed.

An example of the probe immobilized substrate prepared by applying the metal or metal compound on the substrate by the ink-jet method is shown in FIG. 6.

The conductive layer is deposited so that the spots 103 of the liquid containing the metal or metal compound overlap with each other at respective ends as shown in FIG. 6. It is preferable to prepare the composition of the liquid so that the droplets are unnecessarily expanded on the substrate so as to stay at prescribed positions. Since the droplets supplied onto the surface of the substrate is minute, it is effective to allow the discharged liquid to contain the moisture retaining agent in order to prevent the liquid from being evaporated after application. Since the temperature of the liquid increases during discharge particularly in the thermal-jet method, adding the moisture retaining agent and surface tension adjusting agent is important. The solvent for supplying the probe onto the surface of the substrate favorably contains 5 to 10 wt % of urea, 5 to 10 wt % of glycerin, 5 to 10 wt % of thiodiglycol and 1 wt % of acetylene alcohol.

While any method may be used for preparing the solution containing the metal or metal compound for applying the spots 103 of the liquid containing the metal or metal compound so long as the method is able to stably and continuously discharge fine droplets, the solution is favorably prepared by dissolving, for example, an organometallic complex or metallic colloid in an appropriate solvent since the layer is preferably deposited to be a fine particle layer after deposition.

Example of center metals for use in the organometallic complex include metals such as Pt, Pd, Ru, Au, Ag, Cu, Cr, Ta, Fe, W, Zn and Sn. Water or an organic solvent is used for the solvent depending on the solubility of the organometallic complex. However, water is preferable considering reduction of the cost and environmental protection, and the solvent is favorably prepared by dissolving a solvent containing the moisture retaining component and surface tension adjusting component in water.

Examples of the metallic colloid include colloids of the metals such as Pd, Au, Ag and Pt, and a colloid of SnO₂. Solvents of the colloid include water or organic solvents, although they depend on the method for preparing the colloid. Examples of the organic solvent include hexane and pentaethyleneglycol decylether.

The spots 103 of the liquid containing the metal or metal compound may be formed by the ink-jet method at the same time as forming the spots 102 of the probe, or the spots of the liquid may be formed before forming the spots of the probe.

When the spots 103 of the liquid containing the metal or metal compound are formed in advance to forming the spots of the probe, water may be used as the principal solvent of the solution containing the metal or metal compound even when the substrate is subjected to a process for washing with water, since the spots of the liquid containing the metal or metal compound has been previously formed.

Actually, an aqueous solution of a specified organometallic compound or a metallic colloid, or a solution prepared by adding an appropriate moisture retaining agent and surface tension retaining agent in the aqueous colloid solution may be used.

The spots 103 are formed by spontaneous drying or by heating the substrate for evaporating the solvent, after the spots 103 of the liquid containing the metal or metal compound have been discharged. The substrate on which the spots 103 are formed is dried at a high temperature of 200 to 400° C. in order to remove organic components in the complex, although the temperature depends on the material of the substrate and the kind of the organometallic complex, thereby forming the spots 103 comprising fine particles of the metal or metallic compound.

The spots 102 of the probe are formed thereafter by the same method as simultaneously forming the spots by discharging the solution containing the probe followed by allowing the probe to react with the substrate.

Various kinds of metals or metal compounds can be used as marker spots 103 using the method for forming the spots as described above.

The solution containing the metal or metal compound should be devised so that the spots 103 of the metal or metal complex are not removed in the cleaning step when the spots 103 of the solution containing the metal or metal compound are formed in the same step as forming the spots 102 of the probe, considering the cleaning step applied after a reaction of the probe array and target substance after forming both kinds of spots.

In an effective method, the proportion of the moisture retaining agent is reduced to be smaller than the proportion of the solution containing the probe in the solution containing the metal or metal compound. Only the solvent of the solution containing the metal or metal compound is dried during the coupling reaction time between the solution containing the probe and the substrate in order to prevent the spots 103 from being removed in the cleaning step. The proportion of the moisture retaining agent is appropriately selected depending on the principal solvent of the ingredients except the moisture retaining agent.

The embodiment of the probe immobilized substrate with a marker will be described below.

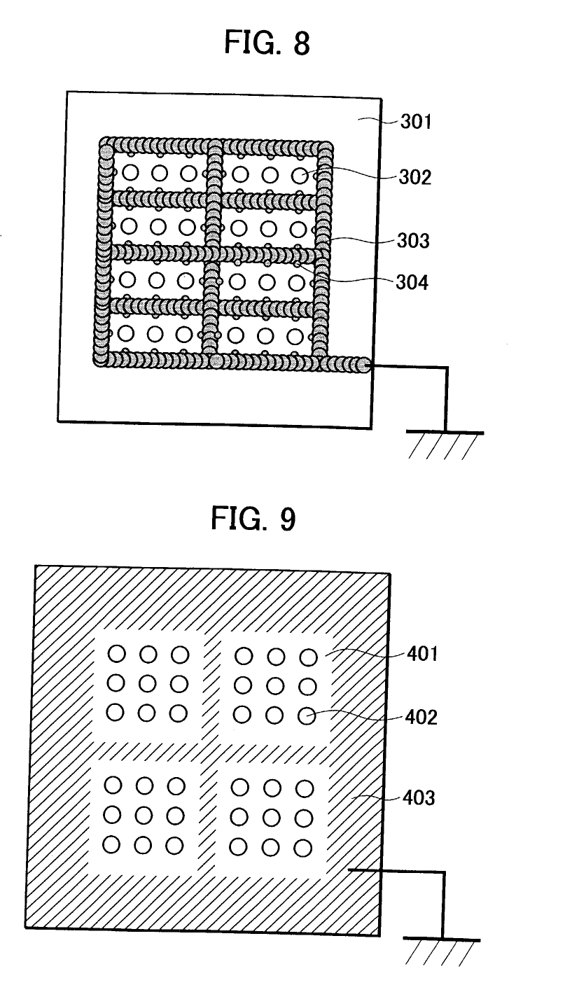

The probe can be readily located by depositing the conductive layer by the ink-jet method while depositing a marker indicating the position of the probe as shown in FIG. 8. In FIG. 8, the reference numeral 301 denotes an insulation substrate, the reference numeral 302 denotes a probe array spot, the reference numeral 303 denotes a conductive layer deposited by the ink-jet method so as to overlap the ends of the dots, and the reference numeral 304 denotes a marker.

The substrate, spots and conductive layer are formed as described above. While the ink-jet method is used for forming the conductive layer in FIG. 8, the method is not restricted thereto, and the sputtering method and printing method may be used as well.

Then, the marker 304 is discharged so as to be electrically connected to the conductive layer 303. The marker is formed to have the same X and Y coordinates as the position where the probe is to be immobilized. Any markers may be used so long as they are visualized with the optical microscope, stereoscopic microscope or CCD camera usually attached to the analyzer. For example, since the conductive layer is detectable with the optical microscope or stereoscopic microscope when the marker is colored by irradiating a visible light, such marker is suitable as the marker for locating the probe array spot 302.

The conductive layer 303 and marker 304 may be formed using the same solution, or using different solutions with each other. However, using the same solution is advantageous since only one kind of the material may be prepared.

On the other hand, using aluminum as the material of the conductive layer while using silver as the marker is favorable for more easily locating the marker in the measurement by X-ray photoelectron spectroscopy (XPS), because aluminum having poor photoelectron emission efficiency emits relatively small amount of photoelectrons while the silver marker emits a lot of photoelectrons.

More prompt measurements are possible by forming the conductive layer 303 and the probe array spots 302 in the same step by omitting positioning between the column or row of the probe spots and the conductive layer 303.

<Analysis Method>

Such conductive layer can be readily deposited into a metal layer or metal compound layer having an arbitrary shape using the ink-jet method, and is effective for in-line inspection in the manufacturing process and for the analysis before shipping.

The analysis method of the probe immobilized substrate of the invention will be described hereinafter.

In an example of the invention, the probe immobilized substrate is analyzed with the X-ray photoelectron spectrometer. The probe immobilized substrate of the invention is introduced into the chamber at first.

Since the probe array spots have a very small diameter from the requirement for disposing various kinds of spots on the substrate so that the number of the spots becomes as many as possible, an X-ray from a monochromatic X-ray source is preferably used. However, since the X-ray beam from the monochromatic X-ray beam is more slender than an X-ray beam from a white X-ray source, the substrate happens to be electrified when the X-ray beam is irradiated onto the probe immobilized substrate formed on the insulation substrate. This is because the surface of the substrate tends to be positively charged due to emission of photoelectrons from the surface of the probe immobilized substrate caused by irradiating the X-ray beam onto the substrate.

The substrate is prevented from being electrified by attaching an electrification protective metal cover on the substrate. The metal cover is manufactured by drilling a part of a thin metal plate, and functions for releasing electric charges accumulated on a part of the insulation substrate by placing it on the insulation substrate and by grounding a part of the metal cover. However, while the hole regions of the metal plate are available for measurements, the other regions cannot be subjected to the measurements when the metal cover is placed on the substrate. Consequently, the efficiency of analysis was largely compromised since the substrate should be taken out of the chamber to displace the metal cover for every measurements of all the probe region.

However, the dots of the metal or metal compound 103 are disposed so that the end of each dot overlaps the end of adjoining dot as shown in FIG. 6 in the prove immobilized substrate of the invention. Since the dots of the metal or metal compound 103 functions like the metal cover by grounding the dots, all the probes can be measured at one stroke without displacing the metal cover, thereby improving the efficiency of the analysis.

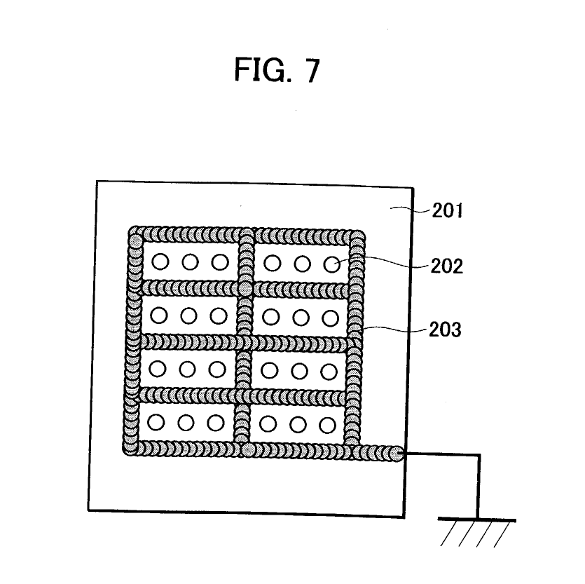

Another embodiment of the probe immobilized substrate will be described below with reference to FIG. 7. The reference numeral 201 denotes an insulation substrate, the reference numeral 202 denotes a probe array, and the reference numeral 203 denotes a conductive layer.

In an example of the analysis method of the invention, the probe immobilized substrate is analyzed with a scanning electron microscope. The probe immobilized substrate is introduced into a chamber at first.

It was impossible to observe the probe immobilized substrate prepared on the insulation substrate such as a glass substrate with the scanning electron microscope due to electrification of the surface of the substrate during observation. When a part or all region of the sample is non-conductive in the observation of the sample configuration under the scanning electron microscope, the sample is electrified by accumulation of electric charges when the electron beam irradiation site is not grounded, and normal observations become impossible due to distortion of the orbit of the electron beam and microscopic images.

However, in the probe immobilized substrate of the invention in which the conductive layer 203 has been disposed around the probe array spots 202, the electric charge accumulated on a part of the surface of the substrate can be released by previously grounding a part of the conductive layer 203, thereby enabling the substrate to be protected from being electrified. Accordingly, it is possible to observe the configuration of the probe immobilized substrate without electrification.

Analysis of the probe immobilized substrate with markers by the scanning electron microscope according to the invention will be described below. The probe immobilized substrate with markers has the structure shown in FIG. 8, and details thereof have been already described above.

When the substrate is introduced into the scanning electron microscope, the overall configuration of the substrate is confirmed at first by a low magnification mode in order to determine the measuring points on the substrate introduced into the chamber. Since the probe array spots are very thin and comprises an organic substance having a low secondary electron emission efficiency without containing any metals having a high secondary electron emission efficiency, it was almost impossible to locate each probe array by the low magnification mode in the conventional probe immobilized substrate comprising only the probe array. However, since the spots of the metal or metal compound 303 are formed on the same lines as the X- and Y-axes in the probe immobilized substrate of the invention, the measuring points can be located by tracing the spots of the metal or metal compound.

While the X-ray photoelectron spectroscopy and scanning electron microscopy have been described as the examples used in the method for analyzing the probe immobilized substrate of the invention, the method is not restricted thereto, and various methods using the electron microanalyzer, Auger electron spectroscopy and SIMS are also effective for analyzing the probe immobilized substrate.

### EXAMPLES

While the invention is described with reference to the examples, the invention is not restricted to these examples.

### Example 1

FIG. 4 is an example of the invention showing a plane view of the probe array in which a plurality of probes having different base sequences with each other are immobilized as the spots on the quartz substrate 401. It is favorable to prepare the probes by dissolving them in a solvent containing a moisture retaining component and surface tension adjusting component. The reference numeral 402 denotes a probe spot. The reference numeral 403 denotes the spot of palladium oxide, which are disposed so as to be able to locate the probe spots 402. Actually, the palladium spots are disposed at the positions corresponding to each column and row of the probe spots 402 arranged in a matrix, thereby enabling each spot 402 to be located.

341 The method for manufacturing the probe immobilized substrate will be described below.

(1) Cleaning of Substrate

The quartz substrate is placed in a one inch square rack, and was immersed in a 1 mol/L (1 N) sodium hydroxide solution previously heated at 60° C. After rinsing with pure water, the substrate was further subjected to a ultrasonic treatment in a vessel filled with pure water for 10 minutes.

(2) Surface Treatment

The substrate was left in a 1% aqueous solution of a silane coupling reagent (trade name KBM603 made by Shin-Etsu Chemical Co.)containing a silane compound (N-β-(aminoethyl)-γ-aminopropyl trimethoxysilane) having amino groups for 20 minutes at room temperature to complete the silane treatment, thereby introducing the amino group on the surface of the substrate. The substrate was washed with pure water followed by drying at 160° C. for 1 hour.

Subsequently, an EMCS solution was prepared by dissolving 2.7 mg of N-(6-Maleimidocaproyloxy)succinimide (made by Dojindo Laboratories, Inc.; abbreviated as EMCS hereinafter) in a 1:1 solution of dimethylsulfoxide (DMSO)/ethanol so that the final concentration becomes 0.3 mg/mL. The quartz substrate subjected to the silane coupling treatment was immersed in this EMCS solution for 30 minutes at room temperature, and the amino group retained on the surface of the quartz substrate by the silane coupling treatment was allowed to react with the carboxyl group in the EMCS solution. After sequentially washing the quartz substrate pulled out of the EMCS solution with a mixed solvent of DMSO and ethanol, and with ethanol, the substrate was dried in a nitrogen atmosphere.

(3) Preparation of Metal Containing Solution

Then, a metal containing solution was prepared. Dissolved in 12 g of water was 0.84 g of a palladium acetate-monoethanolamine complex (abbreviated as a PA-ME complex hereinafter), and a solution prepared by adjusting the viscosity of the solution to 20 centipoise by adding polyvinyl alcohol (abbreviated as PVA hereinafter) was used as an aqueous solution for ink-jet discharge. The PA-ME complex was synthesized as follows.

Palladium acetate (10 g) was suspended in 200 ml of isopropyl alcohol (abbreviated as IPA hereinafter), and the solution was stirred at room temperature for 4 hours after adding 16.6 g of monoethanolamine. IPA was removed by evaporation after completing the reaction, ethanol was added to the solid residue, which was dissolved and filtered, and the PA-ME complex was recrystallized from the filtrate.

(4) Discharge of Metal Compound with BJ Printer, and Immobilization on the Substrate

The solution for bubble-jet discharge prepared as described above was filled in an ink tank of a bubble-jet printer (Trade name BJC 620 made by Canon Inc.), and the tank was attached to a bubble-jet head. The bubble-jet printer (BJC 620 made by Canon Inc.) was reconstructed so as to be compatible with lithographic printing. The metal containing solution was applied on the surface treated quartz substrate as shown in FIG. 1, and the solution was dried.

The PA-ME complex and PVA was decomposed by heating the substrate at 300° C. in the atmosphere in an oven to deposit the decomposition product, thereby forming spots of the metal compound comprising fine palladium particles.

(5) Preparation of DNA Probe

DNAs having the sequence Nos. 1 to 25 (made by BEX Co.) were prepared, and thiol group (SH) was introduced at the 5′-end of DNA using a thiol modifier (made by Glen Research Co.). Subsequently, DNAs were recovered after deprotection by a conventional method, and DNAs purified by high performance liquid chromatography were used in the following experiments.

TABLE 1

| SEQUENCE LENGTH | 18 |
|---|---|
| TYPE OF SEQUENCE | NUCLEIC ACID |
| NUMBER OF STRAND | SINGLE STRAND |
| TOPOLOGY | LINEAR |
| KIND OF SEQUENCE | OTHER NUCLEIC ACID, |
| | SYNTHETIC NUCLEIC ACID |
| SEQUENCE NO. | SEQUENCE |
| 1 | TAGAACCGGAGGCCCATC |
| 2 | GATGGGACTCAAGTTCAT |
| 3 | GATGGGACTCAGGTTCAT |
| 4 | GATGGGACTCACGTTCAT |
| 5 | GATGGGACTCATGTTCAT |
| 6 | GATGGGACTCGAGTTCAT |
| 7 | GATGGGACTCGGGTTCAT |
| 8 | GATGGGACTCGCGTTCAT |
| 9 | GATGGGACTCGTGTTCAT |
| 10 | GATGGGACTCCAGTTCAT |
| 11 | GATGGGACTCCGGTTCAT |
| 12 | GATGGGACTCCCGTTCAT |
| 13 | GATGGGACTCCTGTTCAT |
| 14 | GATGGGACTCTAGTTCAT |
| 15 | ACTCTGGATGGGGTTCAT |
| 16 | GATGGGACTCTCGTTCAT |
| 17 | GATGGGACTCTTGTTCAT |
| 18 | GATGGGGCTCAAGTTCAT |
| 19 | GATGGGGCTCAGGTTCAT |
| 20 | GATGGGGCTCACGTTCAT |
| 21 | GATGGGGCTCATGTTCAT |
| 22 | GATGGGGCTCGAGTTCAT |
| 23 | GATGGGGCTCGGGTTCAT |
| 24 | GATGGGGCTCGCGTTCAT |
| 25 | GATGGGGCTCGTGTTCAT |

(6) Discharge of DNA by BJ printer, and immobilization on the substrate

Twenty five kinds of DNAs were dissolved in the TE solution (a 10 mmol/L aqueous EDTA solution with 1 mmol/L of Tris-HCl(pH 8)) so that the final concentration of DNA becomes 400 mg/mL to prepare respective single strand DNA solution (accurate concentration was calculated from absorption intensity).

An aqueous solution containing 7.5 wt % of glycerin, 7.5 wt % of urea, 7.5 wt % of thiodiglycol and 1 wt % of acetylene glycol (trade name Acetylenol EH made by Kawaken Fine Chemicals Co.) was prepared, and a DNA solution with a final single strand DNA concentration of 8 μmol/L was prepared by adding each DNA above. The solution had a surface tension of 30 to 50 mN/m (30 to 50 dyne/cm), and a viscosity of 1.8 Pa·s (1.8 cps). This solution was filled in an ink tank of a bubble-jet printer (trade name BJC 620 made by Canon Inc.), and the tank was attached to a bubble-jet head. The bubble-jet printer (trade name BJC 620 made by Canon Inc.) as used herein is compatible with lithographic printing, and was reconstructed so as to be able to introduce respective 25 kinds of DNAs. This printer is possible to print with a resolution of 360×720 dpi. Then, a quartz substrate treated as described in (2) above was mounted on this printer, and each solution containing the nucleic acid as a probe was spotted on the quartz substrate. The number of discharged droplets was 225 (15×15) with 25 (5×5) droplets as one unit. The droplets in one unit contain different kinds of DNA probes, respectively, and each unit contains DNAs having the same DNA sequences. The position of each DNA probe was aligned with the position of the palladium oxide dot formed in (4) above so that the position of the DNA probe is not displaced from the position of the palladium oxide dot. After spotting, the quartz substrate was placed in a moisturizing chamber for 30 minutes in order to allow the maleimide group on the surface of the quartz substrate to react with the thiol group at the terminal of the nucleic acid probe. The discharge volume of the DNA probe solution per one discharge operation of the printer was about 24 picoliter.

(7) Inspection of Spot Position

The discharged DNA probe spot prepared in (6) above was located with the optical microscope with reference to the spot of the palladium oxide dot. It was confirmed that each spot of the DNA probe was not displaced, and discharged at the desired position. In addition, each spot did not unnecessarily expanded, and the spot shape was uniform with a mean diameter of about 70 μm.

(8) Blocking Reaction

After completing the reaction between the maleimide group and thiol group, the quartz substrate was washed with a 50 mmol/L of phosphate buffer solution (pH 7.0) containing 1 mol/L of NaCl, and the solution containing excess DNA and not immobilized on the quartz substrate was washed away. Then, the quartz substrate was immersed in a 2% aqueous bovine serum albumin solution for a blocking reaction.

(9) Inspection of Deposited Spot Layer

The probe immobilized substrate after the blocking reaction was washed with pure water, and was observed under a low vacuum scanning electron microscope. The low vacuum scanning electron microscope used was XL 30 SEM made by FEI Co. and the substrate was observed at an acceleration voltage of 15 kV and degree of vacuum of 80 Pa (0.6 Torr).

It was confirmed from the results of observation that each spot of the DNA probe was immobilized at desired positions with no change from the discharge position even after the washing process and blocking reaction. It was also confirmed that no troubles such as deformation by cracks of a part of the spot and recombination of DNA at the region out of the desired regions were caused by the washing process and blocking reaction.

(10) Results

It was very difficult to search the position of the spot in the conventional probe immobilized substrate comprising only the DNA probe spots, since the DNA probe is quite thin and contain only an organic substance having a low secondary electron emission efficiency. It was also very difficult to search the marker spots in the probe immobilized substrate using the fluorescent pigment as a marker.

However, miss-spotting of the spots, satellite spotting, and fogging of the spot by dusts, if any, could be promptly and reliably confirmed by inspection during the manufacturing process with respect to all the DNA probe substrate formed on the probe immobilized substrate of the invention, by providing positions capable of locating the DNA probe as described above, for example by providing the dots of the palladium compound aligned on the same lines as the X- and Y-axes.

In addition, inspection was possible in the manufacturing process without applying any hybridization treatment. Consequently, no products were wasted by sampling for inspection, and all the successful products after inspection could be shipped. This fact is important for quality assurance.

### Comparative Example 1

The same probe immobilized substrate as that in Example 1 was manufactured, except that no metal compound dots comprising palladium oxide were provided. Since colorless and transparent DNA probes were disposed on a colorless and transparent quartz substrate, it was difficult to search the position of the probe. Moreover, since the probe could be hardly located even when miss-spotting was found, and all the heads of the bubble-jet printer were forced to be cleaned, causing a large loss in the manufacturing process.

### Example 2

The same probe immobilized substrate as that in Example 1 was manufactured, except that the metal compound was changed to silver from palladium oxide. The same effect as in Example 1 was obtained in this probe immobilized substrate.

### Example 3

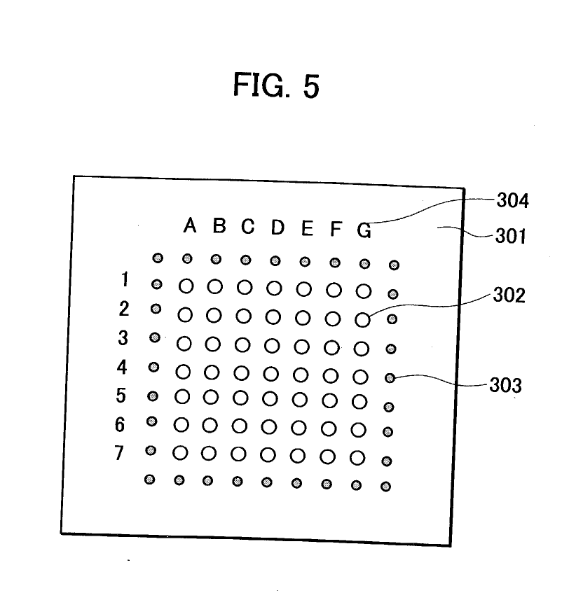

The same probe immobilized substrate as in Example 1 was manufactured, except that X- and Y-addresses 304 were written as shown in FIG. 5, and the discharge volume of the DNA spot was changed to 100 picoliter.

The X- and Y-addresses 304 were written by previously forming the column numbers in the X-direction and row numbers in the Y-direction at the outside of the metal compound dots. The addresses 304 were formed by filling the solution used for printing the marker of the metal compound in another nozzle.

Since the same effect as in Example 1 was obtained while enabling the address of the spot to be previously confirmed by the CCD image and SEM image by the low vacuum scanning electron microscope in this probe immobilized substrate, the nozzle corresponding to abnormal probes could be promptly discovered when some defects of the spot was found.

### Example 4

The metal compound dots and DNA probes were formed on a silicon substrate using the same method as in Example 1, and the substrate was analyzed with the X-ray photoelectron spectrometer.

FIG. 4 is an example of the invention showing a plane view of the probe immobilized substrate in which a plurality of probes having different base sequences with each other are immobilized on the spots on the silicon substrate 401. The reference numeral 402 denotes a spot of the probe. The reference numeral 403 denotes a silver spot, which is disposed so that the probe spot 402 can be located. For example, the silver spots are aligned on the lines corresponding to the column and row of the probe spots 402 aligned as a matrix, and the spot 402 can be located by the silver spots. The diameter of the silver spot and the diameter of the probe spot were designed to be 1 mm, respectively.

The probe immobilized substrate was manufactured by the same method as in Example 1, except that the material of the metal spots was changed to silver. The substrate was placed in the chamber of the X-ray photoelectron spectrometer, and the spot of each probe was analyzed. The X-ray photoelectron spectrometer used was μ-ESCA 250i-XL made by Thermo VG Scientific Co., and monochromatic X-ray from an Al target was used. The filament current of the X-ray tube was 18 mA, and the voltage as 17 kV.

Usually, the probe on the probe immobilized substrate comprising only the DNA probe spots could not be located in the lens barrel, since the DNA probe is a colorless, transparent and very thin layer.

However, the silver spots 403 are disposed at the positions capable of locating the probe spots 402 in the probe immobilized substrate of the invention as shows in FIG. 4. The analysis point can be located from the positions of the silver spots, and each spot can be analyzed in a short period of time by tracing the located positions.

Consequently, each probe could be confirmed to be formed at the desired position on the substrate without causing peeling of the probe and miss-discharge of the droplet. This means that probes sufficiently reliable for use in clinical tests could be certainly formed.

### Example 5

Palladium oxide spots and a protein antibody were prepared on the quartz substrate using the ink-jet method in this example for analysis by TOF-SIMS.

FIG. 1 shows an example of the invention, wherein antibodies corresponding to different proteins were disposed on the quartz substrate. After manufacturing this substrate by the same method as in Example 1, it was placed in the chamber of TOF-SIMS for analysis of each spot of the probe. TOF-SIMS used was TOF-SIMS IV made by ION-TOF Co. The emitter used was made of Ga, and the acceleration voltage and sample current were 25 kV and 2.4 pA, respectively.

It was difficult to locate the probe with the CCD camera in the conventional antibody immobilized substrate, since the probe was transparent and thin. However, since the analysis points can be located by the palladium oxide spots in the substrate of the invention, analysis of each spot was possible within a short period of time. Consequently, the probe was confirmed to be formed as designed with no defective probes.

### Example 6

Palladium oxide dots and DNA probes were formed on a silicon substrata using the method in Example 1, and the dots and probes were observed using a conventional high vacuum scanning electron microscope.

FIG. 4 is a plane view of the probe immobilized substrate showing an example of the invention, wherein a plurality of the DNA probes having different base sequences with each other are immobilized on the silicone substrate 401 as spots. The reference numeral 402 denotes the probe spot, while the reference numeral 403 denotes the palladium oxide spots, which are disposed so as to be able to locate the probe spots 402. Actually, the palladium oxide spots are disposed at the positions corresponding to each column and row of the probe spots 402 arranged as a matrix, and the spots 402 can be located with reference to the palladium oxide spots. The diameters of the DNA probe and palladium oxide probe were adjusted to 70 μm, respectively. The scanning electron microscope used was S-5000H made by Hitachi Co., and the substrate was observed at an acceleration voltage of 1 kV.

The probe immobilized substrate manufactured by the same method as in Example 1 was placed in the lens barrel of the scanning electron microscope, and details of each spot were observed. Usually, it was quite difficult to locate the probe in the lens barrel, since the DNA probe spot is a colorless, transparent and very thin layer.

However, the palladium oxide spots 403 are disposed so as to be able to locate the DNA probe spots 402 in the probe immobilized substrate of the invention as shown in FIG. 4. Consequently, since approximate observation points can be located from the positions of the palladium oxide spots, a good image could be obtained in a short period of time by precisely searching the located points.

Consequently, it was confirmed that each probe had been formed with a design diameter of about 70 μm Since fine structures could be also confirmed with the optical microscope, it was confirmed that the probe array having sufficient reliability for clinical tests had been formed.

### Example 7

FIG. 7 is an example showing a probe array of the invention, wherein spots of proves are immobilized on a quartz substrate 201. The reference numeral 202 denotes a spot of the prove, and the reference numeral 203 denotes a conductive layer comprising palladium oxide, which are electrically connected to an external earth electrode.

The method for manufacturing the probe immobilized substrate will be described below.

A nucleic acid probe array was prepared according to the method described in Japanese Patent Laid-Open No. 11-187900.

(1) Cleaning of the Substrate

A synthetic quartz substrate with a dimension of 25.4 mm×25.4 mm×1 mm was placed in a rack, and was immersed in a ultrasonic detergent (GP-III made by Branson Ultrasonic Co.) diluted to a concentration of 10% with pure water overnight. The substrate was subjected to ultrasonic washing in the detergent for 20 minutes, and the detergent was washed away with water. After rinsing with pure water, the substrate was further subjected to a ultrasonic treatment for 20 minutes in a vessel filled with pure water. Subsequently, the substrate was immersed in an aqueous sodium hydroxide solution (1 mol/L (1N)) previously heated at 80° C. for 10 minutes, followed by washing with water and pure water to submit the substrate to a succeeding process.

(2) Surface Treatment

A 1 wt % aqueous solution of a silane coupling agent having amino groups (N-β-(aminoethyl)-γ-aminopropyl trimethoxysilane; KBM603 made by Shin-Etsu Chemical Co.) was stirred for 2 hours at room temperature to hydrolyze the methoxy group in the silane compound. Then, after immersing the substrate obtained in (1) above in this solution for 1 hour at room temperature, the substrate was washing with pure water followed by drying by blowing nitrogen gas onto both surfaces of the substrate. Subsequently, the substrate was baked in an oven heated at 120° C. for 1 hour to finally introduce the amino group on the surface of the substrate

Then, 2.7 mg of N-(6-Maleimidocaployloxy) succinimide (made by Dojindo Laboratories, Inc., abbreviated as EMCS hereinafter) was dissolved in a 1:1 mixed solvent of dimethylsulfoxide (DMSO) and ethanol so that the concentration becomes 0.3 mg/L. The quartz substrate after a silane coupling treatment was immersed in this EMCS solution for 2 hours at room temperature to allow the amino group that is retained on the surface of the substrate by the silane coupling treatment to react with the succinimide group in the EMCS solution. The maleimide group derived from EMCS is bound on the surface of the substrate by immersion described above. The substrate pulled out of the EMCS solution was sequentially washed with a mixed solvent of DMSO and ethanol, and ethanol, followed by drying by blowing nitrogen gas.

(3) Preparation of Metal Containing Solution

A metal containing solution was then prepared. A palladium acetate-monoethanolamine complex (0.84 g; abbreviated as PA-ME complex hereinafter) was dissolved in 12 g of water, and the viscosity was adjusted to 20 mPa·s (20 centipoise) by adding polyvinyl alcohol (abbreviated as PVA hereinafter). The PA-ME complex was synthesized as follows.

Palladium acetate (10 g) was suspended in 200 ml of isopropyl alcohol (abbreviated as IPA hereinafter), and 16.6 g of monoethanolamine was added to the solution followed by stirring at room temperature for 4 hours. After the reaction, IPA was removed by evaporation, and ethanol was added to the solid residue followed by dissolution of the residue and filtration of the solution. The PA-ME complex was recrystallized from the filtrate.

(4) Discharge of Metal Compound with BJ Printer, and Immobilization on the Substrate

The solution for bubble-jet discharge prepared as described above was filled in an ink tank of a bubble-jet printer (Trade name BJC 620 made by Canon Inc.), and the tank was attached to a bubble-jet head. The bubble-jet printer (BJC 620 made by Canon Inc.) was reconstructed so as to be compatible with lithographic printing. The metal containing solution was applied on the surface treated quartz substrate as shown in FIG. 1, and the solution was dried.

The PA-ME complex and PVA was decomposed by heating the substrate at 300° C. in the atmosphere in an oven to deposit the decomposition product, thereby forming spots of the metal compound comprising fine palladium particles.

(5) Preparation of DNA Probe

A sequence number 26 single strand nucleic acid (40-mer of thymine (T)) was synthesized by requesting to a DNA synthesizing company (BEX Co.). A thiol (SH) group was introduced at the 5′-end of the sequence number 26 single strand DNA using a thiol modifier (Gren Research Laboratories) in the synthetic process. DNA was deprotected and recovered by a conventional method, and was purified by HPLC. A series of process from synthesis to purification was requested to the synthesis company.

Sequence No.: 26 5′-HS—(CH₂)₆—O—PO₂—O-TTTTTTTTTT-TTTTTTTTTT-TTTTTTTTTT-TTTTTTTTTT-3′

(6) Discharge of DNA by Thermal Jet-Printer, and Immobilization on the Substrate

The sequence No. 1 single strand DNA was dissolved in a solution containing 7.5 wt % of glycerin, 7.5 wt % of urea, 7.5 wt % of thiodiglycol and 1 wt % of acetylene alcohol (trade name Acetylenol E100 made by Kawaken Finechemicals Co.) represented by the general formula (I) at a concentration of 8 μmol/L. The printer head BC-50 (made by Canon, Inc.) of a bubble-jet printer BJF-850 (made by Canon, Inc.) using a bubble-jet method as a kind of thermal jet method was reconstructed so as to be able to discharge about 100 μL of solutions, and this head was mounted on a discharge drawing machine reconstructed so as to be able to discharge on the quartz substrate. Hundreds microliter of the DNA solution was injected into the reconstructed tank of the head, and was spotted on the EMCS treated substrate by mounting it on the printer. The amount of discharge for spotting was 4 picoliter/droplet, and the droplets were discharged at a spotting density of 200 dpi within a 10 mm×10 mm area at the center of the substrate, or with a pitch of 127 μm. The diameter of the dot spotted under this condition was about 50 μm.

After completing spotting, the substrate was left in a moisturizing chamber for 30 minutes to allow the maleimide group on the surface of the glass to react with the thiol group at the end of the nucleic acid probe. Then, the substrate was washed with pure water, and stored in pure water.

(7) Inspection of Spot Deposition

The preserved DNA immobilized substrate (DNA chip) was dried by blowing nitrogen gas immediately before observation by the scanning electron microscope, and was further dried in a vacuum desiccator. The chip was adhered on an aluminum sample table after drying, and the conductive layer was grounded by connecting the end of the conductive layer 103 on the surface of the chip to the sample table. The scanning electron microscope used was S-5000H made by Hitachi Co., and the chip was observed at an acceleration voltage of 5 kV.

(8) Results

The chip could be observed at the acceleration voltage of 5 kV without electrification. It was confirmed that each spot of the DNA probe was immobilized at a desired position with no displacement from the position immediately after discharge even after the washing process and blocking reaction. It was also confirmed that no troubles such as deformation by cracking of a part of the spot and recombination of DNA at a position out of the desired position were caused by the washing process and blocking reaction.

The conductive layer was disposed around the spots of the DNA probe in the probe immobilized substrate of the invention. Consequently, miss-spotting of the spot, satellite spots, and fogging of the spot with dusts, if any, could be promptly and reliably confirmed by inspection in the manufacturing process with respect to all the DNA probe immobilized substrate manufactured.

In addition, inspection was possible in the manufacturing process without applying any hybridization treatment. Consequently, no products were wasted by sampling for inspection, and all the successful products after inspection could be shipped. This fact is important for quality assurance.

### Comparative Example 2

The probe immobilized substrate was manufactured by the same method as in Example 1, except that no conductive layer of palladium oxide was provided. The probe was observed by the scanning electron microscope as in Example 1. When the probe was observed under an acceleration voltage of 5 kV as in Example 1, no secondary electron images were obtained due to electrification since the DNA prove was disposed on the insulating quartz substrate. Therefore, the probe immobilized substrate was once taken out of the lens barrel, and the probe was observed by decreasing the acceleration voltage to 1 kV after introducing the substrate into the lens barrel again. However, detailed inspection of the probe was impossible.

### Example 8

The probe immobilized substrate was manufactured by the same method as in Example 7, except that the metal compound was changed from palladium oxide to silver. The same effect as in Example 7 was obtained with respect to this probe immobilized substrate.

### Example 9

The probe was manufactured after depositing the conductive layer by sputtering as shown in FIG. 9. In FIG. 9, the reference numeral 401 denotes a blue sheet glass substrate, the reference numeral 402 denote an array spot, and the reference numeral 403 denotes a conductive layer. The manufacturing method was the same as in Example 1 except the conductive layer. The same effect as in Example 1 was obtained by observation with the scanning electron microscope by the same method as in example 7.

### Example 10

The probe was manufactured by providing markers electrically connected to the conductive layer as shown in FIG. 8. In FIG. 8, the reference numeral 301 denotes a blue sheet glass substrate, the reference numeral 302 denote a probe array spot, the reference numeral 303 denotes a conductive layer, and the reference numeral 304 denotes a marker. The conductive layer 303 was formed as a continuous layer by the ink-jet method so that the ends of the circular palladium oxide layers overlap with each other. The tip of the continuous conductive layer was connected to an earth terminal to electrically ground the layer. The marker 304 comprises palladium spots, which are disposed so as to be able to locate the probe spot 302. Actually, the markers are aligned at the positions corresponding to the columns and rows of the probe spots 302 arranged as a matrix, thereby enabling the spots 302 to be located.

The palladium oxide layer was formed by the ink-jet method, and the probe except the marker was manufactured by the same method as in Example 1.

The probe immobilized substrate was observed with the scanning electron microscope by the same method as in Example 1, obtaining the same effect as in Example 7.

Usually, the position of the spots of the DNA probe could be hardly confirmed in the lens barrel since the spots are colorless, transparent and very thin layer having a low secondary electron emission efficiency. However, the positions of the probe array spots 302 could be searched with reference to the positions of the markers in the probe immobilized substrate of the invention in which the spots 303 of palladium oxide were disposed at the positions capable of locating the spots 302 of the probe as shown in FIG. 8. Consequently, the time for searching the positions of the probe array spots having a low secondary electron emission efficiency as was required in the conventional example could be saved, and good images could be obtained within a short period of time by precisely observing the located points, thereby enabling efficiency of analysis to be improved.

Consequently, it was confirmed that each prove was uniformly formed with a diameter of about 70 μm. The probe array formed was confirmed to be sufficiently reliable for clinical tests.

### Example 11

Dots of the metal compound and DNA probes were formed on the quartz substrate in this example using the method in Example 7, and the probe was analyzed with the X-ray photoelectron spectrometer.

FIG. 11 is a plane view of the probe immobilized substrate, wherein a plurality of probes having different base sequences with each other are immobilized as spots on the quartz substrate 601. The reference numeral 602 denotes a probe spot, and the reference numeral 603 denotes a silver spot, which is disposed so as to be able to locate the probe spot 602. Actually, the silver spots are aligned at the positions corresponding to the columns and rows of the probe spots 602 arranged as a matrix, thereby enabling the spots 602 to be located. The design diameters of the probe and silver spots were 1 mm, respectively.

After manufacturing the probe immobilized substrate by the same method as in Example 5 except that the material of the metal spot was changed to silver, the substrate was placed in the chamber of the X-ray photoelectron spectrometer, and the spots in each probe were analyzed. The X-ray photoelectron spectrometer used was μ-ESCA 250i-XL made by Thermo VG Scientific Co. using a monochromatic X-ray from Al. The filament current and voltage of the X-ray generator were 18 mA and 17 kV, respectively.

Usually, a metal mask was placed on the sample for protecting from electrification during the measurement in the X-ray photoelectron spectrometric measurement of the probe immobilized substrate comprising only the DNA probe spots, and a neutralizing electron gun was used together.

In the case above, the metal mask should be displaced little by little depending on the position of the probe array, in order to analyze the positions out of the openings of the metal mask.

However, the probe immobilized array of the invention shown in FIG. 11 could be measured without electrification since the conductive layer plays a role as the metal mask.

Consequently, it was confirmed that each probe was formed at the desired position on the substrate without peeling and miss-discharge of the probe. Accordingly, it was confirmed that a probe array sufficiently reliable for use in clinical tests has been formed.

What is claimed is:

1. A probe immobilized substrate on which a probe capable of specifically binding to a target substance is immobilized to a first position on the surface of the substrate,
a metal or a metal compound being disposed at a second position capable of locating the first position with relation to the first position.

2. The probe immobilized substrate according to claim 1, wherein the metal or the metal compound is colored.

3. The probe immobilized substrate according to claim 1, wherein the metal or the metal compound is formed into a fine particle layer.

4. The probe immobilized substrate according to claim 3, wherein the particle diameter of the particles forming the fine particle layer is 1 μm or less.

5. A method for manufacturing a probe immobilized substrate comprising the steps of:
supplying a liquid containing a probe capable of specifically binding to a target substance on a first position of the surface of a substrate; and
disposing a metal or a metal compound at a second position capable of locating the first position with a special relation to the first position,
the probe being supplied after disposing the metal or the metal compound.

6. A method for manufacturing a probe immobilized substrate comprising the steps of:
supplying a liquid containing a probe capable of specifically binding to a target substance on a first position of the surface of a substrate; and
disposing a metal or a metal compound at a second position capable of locating the first position with a special relation to the first position,
the metal or the metal compound being supplied onto the surface of the probe immobilized substrate at the same time as supplying the liquid containing the probe to the surface of the substrate.

7. A method for analyzing a probe array having probes capable of specifically binding to target substances that may be potentially contained in a test sample as a plurality of spots being independent with each other, comprising the steps of:
detecting a marker comprising the metal or metal compound so as to be able to locate each spot on the surface of the substrate; and
locating each spot based on the position of the marker.

8. The method for analyzing the probe array according to claim 7, wherein the analyzer used for the probe array analysis is a scanning electron microscope, an X-ray photoelectron spectrometer, an atomic force microscope, or a secondary ion mass spectrometer.

9. A method for detecting a probe and a target material, if any, in any one of the spots by allowing each spot of the probe array having the probes that specifically bind to a target substance that may be potentially contained in a test sample as a plurality of mutually independent spots to contact the test sample, comprising the step of:
locating the spot containing a reaction product based on a metal or a metal compound when the reaction product is detected to exist.

10. A probe immobilized substrate comprising a probe capable of specifically binding to a target substance immobilized on the surface of a substrate having at least an insulating surface,
a conductive layer being flush with at least the surface of the probe.

11. The probe immobilized substrate according to claim 10, wherein the conductive layer has a volume resistivity of 1×107 Ω·cm or less.

12. The probe immobilized substrate according to claim 10, wherein the conductive layer comprises a metal, a metal compound, carbon, or fine particles.

13. The probe immobilized substrate according to claim 10, wherein the substrate having the insulating surface comprises an insulating layer disposed on the surface of the conductive substrate.

14. A method for manufacturing a probe immobilized substrate comprising the steps of:
supplying a liquid containing a probe capable of specifically binding to a target substance; and
disposing a conductive layer on the surface of the substrate,
the probe being supplied after disposing the conductive layer.

15. A method for manufacturing a probe immobilized substrate comprising the steps of:
supplying a liquid containing a probe capable of specifically binding to a target substance; and
disposing a conductive layer on the surface of the substrate,
the conductive layer being disposed at the same time as supplying the liquid containing the probe onto the surface of the substrate.

16. The method for manufacturing the probe immobilized substrate according to claim 15, wherein the metal compound is an organic metal complex.

17. The method for manufacturing the probe immobilized substrate according to claim 15, wherein the metal or the metal compound is in the form of fine particles.

18. A method for analyzing a probe array comprising probes capable of specifically binding to a target substance that may be potentially contained in a test sample as a plurality of spots being independent of each other,
wherein the probe is analyzed after disposing the conductive layer on the surface of the substrate followed by electrically connecting the conductive layer to the ground.

19. The method for analyzing the probe array according to claim 18, wherein the analyzer used for analyzing the probe array is a scanning electron microscope, a X-ray photoelectron spectrometer, a secondary ion mass spectrometer or a time-of-flight secondary ion mass spectrometer.


SEQUENCE LISTING (26 sequences)

SEQ ID NO: 1 - DNA, 18 bp, Artificial Sequence
  Synthesized
atgaaccggaggcccatc

SEQ ID NO: 2 - DNA, 18 bp, Artificial Sequence
  Synthesized
gatgggactcaagttcat

SEQ ID NO: 3 - DNA, 18 bp, Artificial Sequence
  Synthesized
gatgggactcaggttcat

SEQ ID NO: 4 - DNA, 18 bp, Artificial Sequence
  Synthesized
gatgggactcacgttcat

SEQ ID NO: 5 - DNA, 18 bp, Artificial Sequence
  Synthesized
gatgggactcatgttcat

SEQ ID NO: 6 - DNA, 18 bp, Artificial Sequence
  Synthesized
gatgggactcgagttcat

SEQ ID NO: 7 - DNA, 18 bp, Artificial Sequence
  Synthesized
gatgggactcgggttcat

SEQ ID NO: 8 - DNA, 18 bp, Artificial Sequence
  Synthesized
gatgggactcgcgttcat

SEQ ID NO: 9 - DNA, 18 bp, Artificial Sequence
  Synthesized
gatgggactcgtgttcat

SEQ ID NO: 10 - DNA, 18 bp, Artificial Sequence
  Synthesized
gatgggactccagttcat

SEQ ID NO: 11 - DNA, 18 bp, Artificial Sequence
  Synthesized
gatgggactccggttcat

SEQ ID NO: 12 - DNA, 18 bp, Artificial Sequence
  Synthesized
gatgggactcccgttcat

SEQ ID NO: 13 - DNA, 18 bp, Artificial Sequence
  Synthesized
gatgggactcctgttcat

SEQ ID NO: 14 - DNA, 18 bp, Artificial Sequence
  Synthesized
gatgggactctagttcat

SEQ ID NO: 15 - DNA, 18 bp, Artificial Sequence
  Synthesized
gatgggactctggttcat

SEQ ID NO: 16 - DNA, 18 bp, Artificial Sequence
  Synthesized
gatgggactctcgttcat

SEQ ID NO: 17 - DNA, 18 bp, Artificial Sequence
  Synthesized
gatgggactcttgttcat

SEQ ID NO: 18 - DNA, 18 bp, Artificial Sequence
  Synthesized
gatggggctcaagttcat

SEQ ID NO: 19 - DNA, 18 bp, Artificial Sequence
  Synthesized
gatggggctcaggttcat

SEQ ID NO: 20 - DNA, 18 bp, Artificial Sequence
  Synthesized
gatggggctcacgttcat

SEQ ID NO: 21 - DNA, 18 bp, Artificial Sequence
  Synthesized
gatggggctcatgttcat

SEQ ID NO: 22 - DNA, 18 bp, Artificial Sequence
  Synthesized
gatggggctcgagttcat

SEQ ID NO: 23 - DNA, 18 bp, Artificial Sequence
  Synthesized
gatggggctcgggttcat

SEQ ID NO: 24 - DNA, 18 bp, Artificial Sequence
  Synthesized
gatggggctcgcgttcat

SEQ ID NO: 25 - DNA, 18 bp, Artificial Sequence
  Synthesized
gatggggctcgtgttcat

SEQ ID NO: 26 - DNA, 40 bp, Artificial Sequence
  Synthesized
tttttttttttttttttttttttttttttttttttttttt